(19) United States
(12) Patent Application Publication
(10) Pub. No.: US 2012/0122636 A1
(43) Pub. Date: May 17, 2012

(54) INFRARED HEAT EXERCISE PAD

(76) Inventor: DAVID FLOYD SHURTLEFF, LAKEWOOD, CO (US)

(21) Appl. No.: 12/944,628

(22) Filed: Nov. 11, 2010

(51) Int. Cl. A63B 26/00; A61F 7/00

(52) U.S. Cl. 482/142; 607/96

(57) ABSTRACT

A flexible infrared heat exercise pad provides a warming surface through the use of infrared energy transmissions from the pad to the user. The infrared heat exercise pad includes a top layer and a bottom layer of cloth material, sandwiching a coiled heating wire having an upper and lower foil layer. A layer of padding, such as foam or cotton-based padding, is provided. A power source is connected to the coiled heating wire to provide a current through the wire. In response to the current, infrared heat is created and, by coupling through the upper and lower foil layers, provides a consistent heat emission from the surfaces of the pad. The heat level can be set ranging from low to high, and the duration of heating can be set to accommodate workouts having varying periods of use, or for the first portion of a workout, for example.

### FIELD OF THE INVENTION

The present invention relates generally to exercise equipment. The present invention is more particularly, though not exclusively, useful as a mat for floor-based exercises, such as yoga. The present invention is particularly suited for use as a mat for use in conjunction with yoga or other exercises which require prolonged contact with the floor.

### BACKGROUND OF THE INVENTION

Yoga has been practiced for centuries. Throughout this practice, in order to provide some level of comfort to the athlete, a variety of mats have been used. In some cases, the yoga mat is a woven bamboo mat intended to simply create a physical barrier between the yoga practitioner and the floor. In other cases, the mat consists of a padded foam panel that provides some measure of cushion between the practitioner and the often concrete floor. Yoga, like many other floor-based exercises, can often result in a practitioner becoming stiff due to muscle tightness and fatigue. In order to minimize this tightness and fatigue, the room where yoga is practiced is often heated. However, when heating sufficiently to minimize the muscle tightness and fatigue, the heat is often too high for the yoga practitioner to be comfortable. As a result, either the room is cooled and the practitioner is chilled, or the room is heated, and the practitioner is uncomfortable.

Various types of exercise mats used by individuals while executing yoga positions are known in the prior art. For instance, U.S. PreGrant Publication No. 2008/0116190 to Lewin discloses a heated body mat realized by an assembly covered by a protective enclosure. The assembly has a major dimension (e.g., length) and includes a heat reflecting layer disposed between a top foam panel and a bottom foam panel. A resistive heater element is arranged in a single loop that provides coverage over a substantial part of the area of the mat. A number of thermostats are integrated as part of the loop and arranged in a series configuration being spaced apart along the major dimension of the assembly. Each one of the thermostats is normally closed and opens at one or more predetermined threshold temperatures to thereby open the current path loop realized by the resistive heater element. In use, electric power is supplied to the resistive heater element to generate heat that is emitted from the mat.

U.S. PreGrant Publication No. 2007/0088232 to Corradini discloses another mat for yoga and Thai massage configured so as to accommodate the full body length of a practitioner in any direction with respect to the mat, and is provided with functional indicia to facilitate the instruction and/or the practice of yoga, Thai massage art, and other therapeutic practices.

U.S. PreGrant Publication No. 2005/0192158 to Edwards discloses a composite yoga mat having a fabric sheet with a frictional material and/or loop holes to provide support, a stable surface, and moisture absorption for the practice of yoga.

U.S. PreGrant Publication No. 2004/0250346 to Vasishth discloses an anti-slip, multi-layer exercise mat that includes an upper fabric layer, a lower pliable foam layer, and an intermediate adhesive such that during use, the mat provides the athlete with safety and comfort and easy transport or storage.

U.S. Pat. No. 7,657,952 to Hutchison discloses a mat supplemental to an exercise mat and includes a cushion portion for assisting in execution and/or maintaining of a select body position (e.g., a stretch, yoga pose, or Pilates pose). The supplemental mat may include a strap detachable from the cushion portion to secure the cushion portion to the traditional exercise mat, thereby causing the supplemental mat to also serve as a carrier for the traditional exercise mat.

U.S. Pat. No. 7,108,635 to Howlett-Campanella discloses a yoga mat having a symmetrical body placement guide on an upper surface of the mat. The body placement guide is configured to aid a yoga practitioner to properly align the practitioner's body during yoga postures. The body placement guide includes a patterned design defining a longitudinal axis substantially extending the length and bisecting the upper surface of the mat. The patterned design also defines a transverse axis bisecting the longitudinal axis, and together, the longitudinal axis and transverse axis define four equal quadrants, each being a mirror image of the pattern design of the two adjoining quadrants.

U.S. Pat. No. 7,007,322 to Alane discloses a yoga mat that may be rolled up tightly and then held closed by straps that wrap around and encircle the rolled up mat. The straps are affixed to the mat by the ends thereof being sewn to the edge of mat under an attachment strip, which fits along one entire edge of the mat. A shoulder strap is also attached under the attachment strip between the two straps, so that the mat can be carried over the shoulder.

In light of the above, it would be advantageous to provide a mat for use in conjunction with floor exercises that provides some heating benefit to the practitioner without overheating. It would also be advantageous to provide a heating pad that provides heat in a manner that warms the practitioner without causing undue heat stress on his or her body. It would be further advantageous to provide a pad suitable for use, and reuse, while providing for the easy transportation and storage of the pad.

### SUMMARY OF THE INVENTION

The present invention is a flexible infrared heat exercise pad that provides a warming surface through the use of infrared energy transmissions from the pad to the user. The infrared heat exercise pad includes a top layer and a bottom layer of cloth material, sandwiching a coiled heating wire having an upper and lower foil layer. A layer of padding, such as foam or cotton-based padding is provided.

A power source is connected to the coiled heating wire to provide a current through the wire. In response to the current, infrared heat is created and, by coupling through the upper and lower foil layers, provides a consistent heat emission from the surfaces of the pad. The heat level can be set ranging from low to high, and the duration of heating can be set to accommodate workouts having varying periods of use, or for the first portion of a workout, for example.

The structure of the present invention provides an infrared heat exercise pad that can be repeatedly rolled up for storage and placed on the floor for use.

### BRIEF DESCRIPTION OF THE DRAWING

The nature, objects, and advantages of the present invention will become more apparent to those skilled in the art after considering the following detailed description in connection with the accompanying drawings, in which like reference numerals designate like parts throughout, and wherein:

FIG. 1 is a perspective view of the infrared heat exercise pad of the present invention showing the pad, power control device, and power supply;

FIG. 2 is an exploded view of the infrared heat exercise pad of the present invention showing the upper material layer, the lower material layer, a coiled heating wire having an upper and lower foil layer, and a padding layer, with an adhesive on the inner surfaces of the upper and lower material layers, all bound together with a perimetric binding;

FIG. 3 is a top plan view of the power control device of the infrared heat exercise pad of the present invention showing a display, a trio of mode indicators, a mode select and start button, along with a (+) and (−) selection for changing the settings selected;

FIG. 4 is a cross-sectional view of the infrared heat exercise pad of the present invention showing the arrangement of the various layers within the device; and

FIG. 5 is an exploded view of the cross-sectional view of FIG. 4, showing the upper and lower material layers, the foil layers surrounding the coiled heating wire having an insulative covering, and a padding layer.

### DETAILED DESCRIPTION

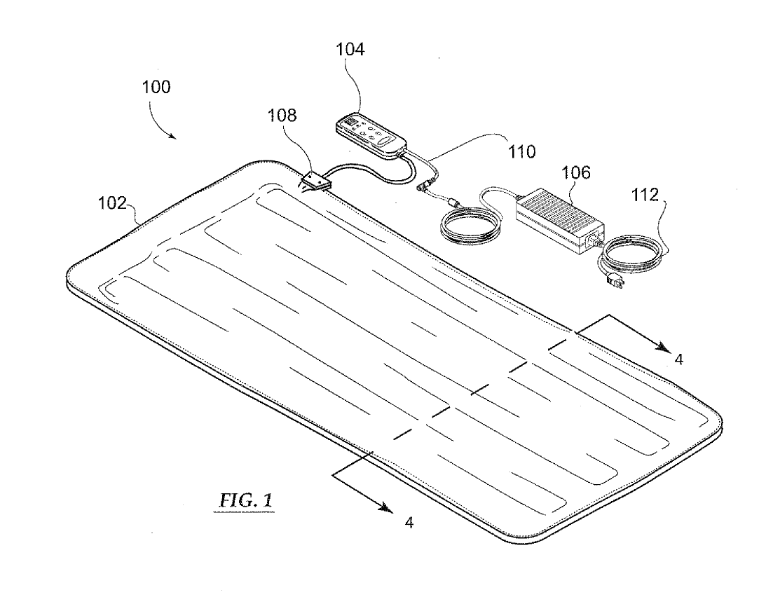

Referring initially to FIG. 1, a perspective view of the infrared heat exercise pad of the present invention is shown and generally designated 100. Pad 100 includes a pad 102, a power control device 104, and a power supply 106. Pad 102 includes a connection 108 for attachment to power control device 104, which receives its input power from cable 110 from power supply 106. AC power cord 112 provides the power for power supply 106. As is known in the art, power supply 106 receives alternating current (AC) power from a wall outlet, and generates a variety of direct current (DC) voltages. These DC voltages are provided to the power control device 104. In response, the power control device 104 provides a DC current to the pad 102 which in turn generates far infrared radiation that emanates from the pad 102. The DC voltages can be a steady state voltage, such as a set DC voltage level, or the voltage can be pulsed to provide an intermittent voltage with varying duty cycle depending on the amount of heat desired. In a preferred embodiment, the DC voltages may be pulse-width modulated (PWM) to achieve an overall heating requirement, yet minimize the steady state power requirements for the device.

In alternative embodiments, power supply 106 may generate AC power to power control device 104. In such circumstances, power control device will in turn provide AC power to pad 102 to be used to generate the necessary heat.

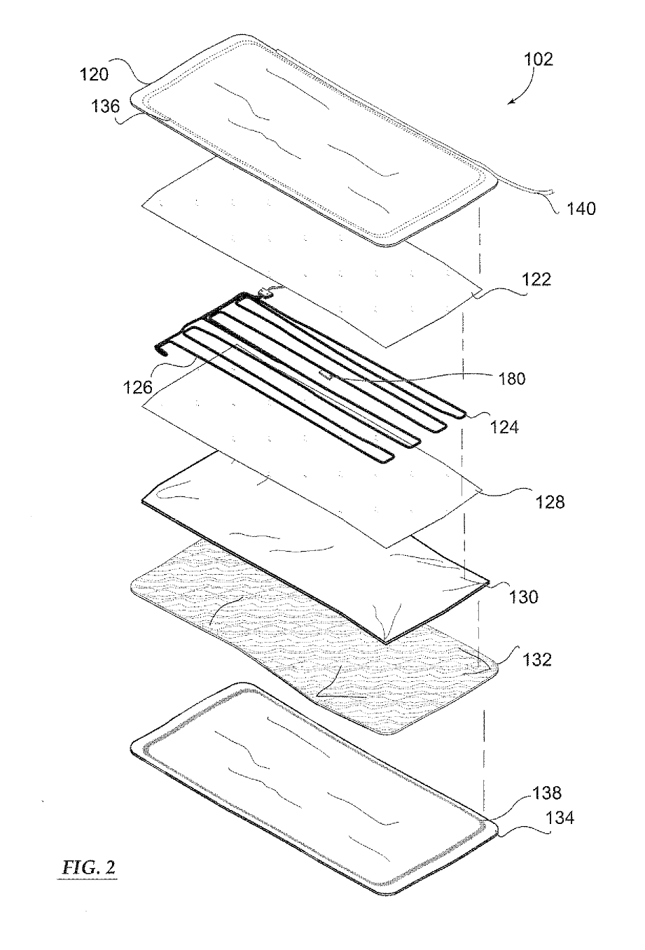

Referring to FIG. 2, an exploded view of the infrared heat exercise pad of the present invention is shown. Pad 102 includes an upper material layer 120 and an intermediate material layer 130. In a preferred embodiment, layers 120 and 130 are made from a heavy canvas material. This material is well suited for application in the present invention because of its durability and ability for heat to pass through the material. It is to be appreciated, however, that other materials may be used without departing from the present invention. Such other materials, for instance, may include but not limited to cotton, terry cloth, nylon, or other synthetic materials known in the art. It is also to be appreciated that while the upper material layer 120 and the intermediate material layer 130 are shown to be the same materials in the Figures, these layers may be of dissimilar materials.

A coiled heating wire 124 is positioned between an upper foil layer 122 and a lower foil layer 128. Foil layers 122 and 128 serve to distribute the heat that is generated from the coiled heating wire 124 that follows a serpentine path between the two foil layers. For example, as the foil layers cover the entirety of the coiled heating wire 124, as the DC current passes through the heating wire 124, far infrared heat is emitted. Far Infrared (FIR) radiation is typically in the 50-1000 μm range, and is generated through the electrical excitation of the coiled heating wire 124.

In the infrared heat exercise pad of the present invention, infrared radiation is used as a deliberate heating source. Specifically, it is used to heat the occupants of the pad, not the surrounding environment or the air around them. This method of heating is a safe method of natural health care and physiotherapy.

In the infrared heat exercise pad of the present invention, the foil layers 122 and 128 encompass the coiled hearing wire 124 so that the radiation emanating from the wire 124 is electromagnetically coupled to the foil layers. As a result, the heat is then retransmitted from the foil layers. Since the foil layers 122 and 128 are continuous across the entire pad 102, the heat is continuous across the surface of the pad 102. Specifically, instead of having traditional “hot spots” that occur with traditional electric blankets or heating pads, the use of FIR electromagnetically generated heat, and the coupling to the foil layers 122 and 128, provide continuous heat across the surface. Moreover, by providing a consistent heating, instead of “hot spot” heating, the risk of burns is lower.

A padding layer 132 layer is provided beneath the intermediate material layer 130 to provide some padding between the floor and the upper material layer 120. In a preferred embodiment, padding layer 132 is made from cotton padding, however, it is to be appreciated that this material is merely exemplary of a preferred embodiment. Other materials may be used to construct padding layer 132. An alternative embodiment of padding layer 132 may be made from sheet foam to provide a softer pad 102, and would be particularly useful when using the present invention on concrete or hard floor surfaces.

A lower material layer 134 is beneath padding layer 132. In a preferred embodiment, lower material layer 134 is from the same material as upper layer 120 and intermediate layer 130. However, other materials are fully contemplated. For instance, the lower material layer 134 may be made from a non-skid material to prevent slippage of the pad 102 on a floor, or a waterproof material to prevent moisture from passing into the pad 102.

An adhesive, such as an epoxy, 136 and 138 are provided on the inner surfaces of the upper and lower material layers 120 and 134. This adhesive, as shown, will capture the intermediate layers within the pad 102. Specifically, when the upper layer 120 and the lower layer 134 are brought together, adhesive 136 binds to adhesive 138 to sandwich the intermediate layers (foil 122, wire 124, foil 128, intermediate layer 130 and padding 132) securely in position within the pad 102, and prevents the shifting or bunching of the various components of the panel 102.

Once all layers are positioned adjacent each other, the layers are all bound together with a perimetric binding 140. This binding, in a preferred embodiment, is sewn to the pad 102 through the perimeters of upper layer 120, padding 132 and lower layer 134. This binding provides for a flexible pad 102, and prevents the various layers from separating during prolonged periods of use.

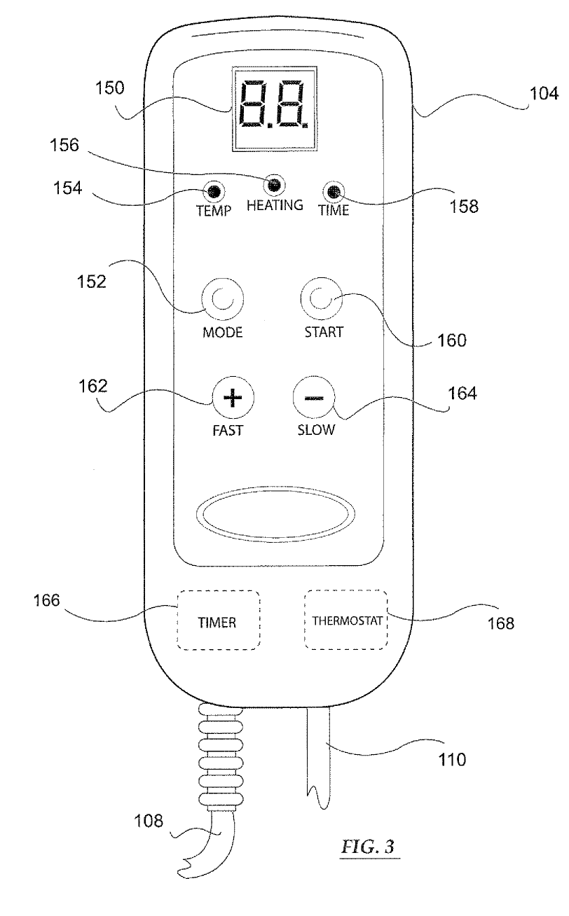

FIG. 3 is a top plan view of the power control device 104 of the infrared heat exercise pad 100 of the present invention. Power control device 104 includes an alphanumeric display 150. Display 150 provides visual feedback to the user of the various time settings and temperature settings of operation. Three mode indicators (temp 154, heating 156, and time 158) are each selected by the repeated pressing of mode select button 152. A start button 160 provides the user with the ability to start the heating cycle for the pad 102 following the programming and setting of the various modes selections. A (+) button 162 and a (−) button 164 are provided to allow the user to increase or decrease the settings in the mode selections.

In use, the user of the infrared heat exercise pad of the present invention 100 presses the mode button 152 to select the TEMP mode, and the temp indicator 154 lights. Display 150 displays a default temperature setting, and by pressing the (+) or (−) buttons 162 and 164, the user may raise or lower the temperature settings. Once satisfied with the temperature setting, the mode button 152 is pressed to select the HEATING mode and the heating indicator 156 light. The heating level is displayed in display 150 and can be adjusted by pressing the (+) or (−) buttons 162 and 164. Once satisfied with the heating mode setting, the mode button 152 is pressed to select the TIME mode and the time indicator 158 lights. The default time setting can be adjusted by pressing the (+) or (−) buttons 162 and 164 to select the desired period of operation. In a preferred application, this would be set to the duration of the yoga class, for example.

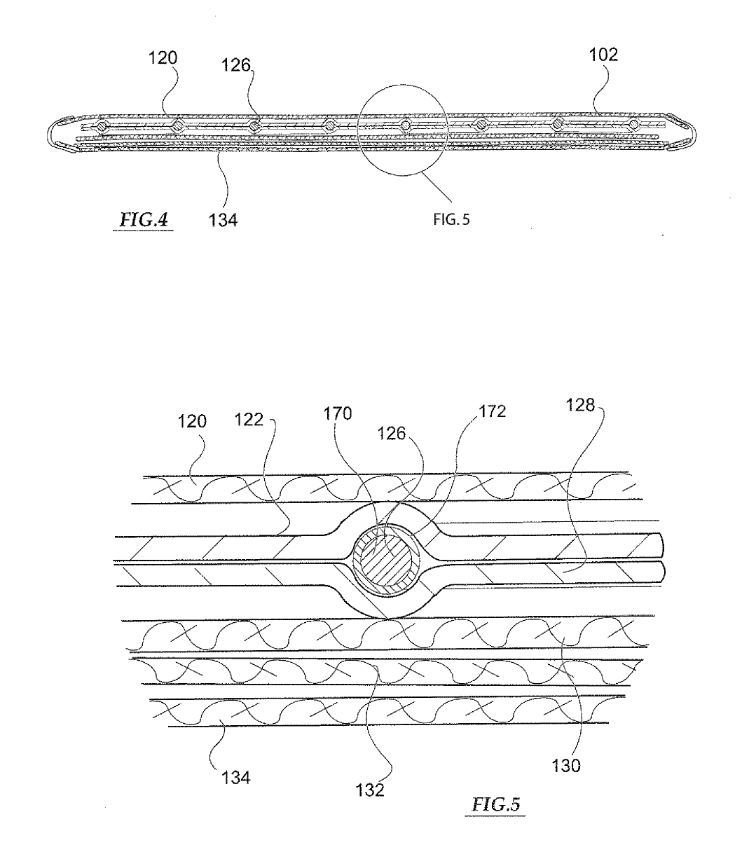

FIG. 4 is a cross-sectional view of the infrared heat exercise pad 100 of the present invention as shown by line 4-4 of FIG. 1. This Figure clearly demonstrates the layered arrangement of the various layers within the pad 102. Specifically, the positioning of upper layer 120, intermediate layer 130 and lower layer 134 show the overall structure of the pad. An enlarged cross-sectional view is shown in FIG. 5. From this view, all layers of the pad 102 are easily seen.

FIG. 5 shows the upper layer 120 covering the upper foil layer 122. The flexible nature of the foil layer 122 allows the foil to closely surround wire 126. Wire 126 includes a central conductor 170, and an outer insulation 172. In a preferred embodiment, central conductor 170 of wire 126 is made from a carbon heating wire. The carbon heating wire is flexible, and provides a known resistance per unit length. This resistance, when subjected to an electrical current passing through the wire, creates a heat radiation in the far infrared wavelength. Insulation 172 is made from an insulated silica gel, and while providing electrical insulation from wire 126, provides little or no resistance to the emanation of the far infrared heat generated by the wire. This preferred embodiment provides for a flexible wire 126 that can be repeatedly used and stored without damage to either the conductor 170 or insulation 172. It is also to be appreciated that these materials are merely exemplary, and that other materials known in the art can be used without departing from the present invention. Lower foil layer 128 is positioned beneath the wire 126 and on top of intermediate layer 130. The padding layer 132 is between the intermediate layer 130 and the lower layer 134.

In addition to the various layers shown herein, a spray adhesive may be used between the various layers to facilitate manufacturing and to ensure longevity of the device. This adhesive may be used in specific areas, or may be applied liberally across the surface of each layer.

### Alternative Embodiments

While there have been shown what are presently considered to be preferred embodiments of the present invention, it will be apparent to those skilled in the art that various changes and modifications can be made herein without departing from the scope and spirit of the invention. For instance, referring generally to FIG. 2, there is disclosed an exploded perspective view of an embodiment of the infrared heat exercise pad of the present invention. In an alternative embodiment, the pad may be constructed with one or more temperature sensors 180 for providing temperature feedback to power control device 104. Alternatively, multiple temperature sensors 180 may be provided to enable the power control device 104 to control the temperature through separate zones of the heating coil 126. In this manner, power control device may include a thermostat 168 (shown in phantom in FIG. 3) for the portion of the pad near the temperature sensor, and can maintain a temperature through the selective increasing or decreasing of the electrical current passed through that portion of the pad 102.

Also, the infrared heat exercise pad 102 includes a bottom layer or ply 134 and a top layer or ply 120 which can be made of rubber or a foam plastic with sufficient friction to prevent a user of the mat from sliding on the mat or the mat from sliding on the floor. The top and bottom plies of the mat may be composed of a flexible, wear-resistant, slide-proof material such as, for example, a soft sponge like rubber material. In those instances, where a foam plastic such as PVC is used for both the top and bottom plies, the top and bottom surfaces of the two plies can be coated with a high friction material such as rubber to prevent sliding.

The top and bottom plies 120 and 134 can, in a preferred embodiment, have a thickness of between one eighth and one quarter of an inch. Thus, the total thickness of the top and bottom plies of the yoga mat is between one-quarter of an inch and one half of an inch. A preferred thickness of the top and bottom plies is about one quarter of an inch, more or less. In a preferred embodiment, the infrared heat exercise pad has a total thickness of between one quarter and one half of an inch depending on the thicknesses of the top and bottom plies.

The thickness of the center ply consists of heating wires 126 located between a layer of aluminum foil 122 and a silver pad 128 does not significantly increase the thickness of the yoga mat. The mat can have a length of between sixty and seventy two inches and a width of between twenty four inches and thirty inches, more or less, to provide a mat that is of sufficient size for use by an adult. In addition, the mat should be free of Phthalates, have both surfaces in at least one of a variety of colors, and include a focal point icon for the user to focus on.

Referring back to FIG. 1, heating coil 126 may be formed to have an electrical circuit with multiple, such as between one and four, separately controlled heating elements and is sandwiched between an upper aluminum foil 122 layer, and a lower silver pad layer 128 to form a center heat assembly which is attached to the top surface of the bottom layer 138 of the infrared heat exercise pad 102.

The heat generating resistance wires are flexible and may be coated with a flexible electrical insulating layer of PVC or the like. In this embodiment where there are multiple zones, the multiple individual heating elements provide multiple zones which are connected in parallel with each other and each zone is connected to power control device 104. in an embodiment where there is only a single heating zone, the heating coils within the heating element can be connected in parallel or in series to heat the infrared heat exercise pad to a uniform temperature.

In an embodiment of the infrared heat exercise pad where there are multiple zones, the zones can provide heat to different areas of equal size on the mat or each zone can heat an area of a different size. For example, the lower section of the mat which is normally under a user's legs and feet may be heated by two zones where the zone for warming the mat under the user's legs will occupy an area that is larger than the zone that is provided for warming the mat under the user's feet. In addition, in those instances where the user has cold feet, the warming zone located under the user's feet can be set to provide more heat than the warming zone located under the user's legs. In a similar manner, the zone at the top of the mat may be set to provide more heat than the zone which is under the user's torso where he/she sits to help avoid over heating the user and possibly causing him/her to perspire unnecessarily.

Each heating zone may be connected to its own controllable thermostat within the power control device 104 which a user can set to a level which is most comfortable to the user's requirements. In addition to a thermostat in each zone, a timer 166 (shown in phantom in FIG. 3) within the power control device 104 is controlled by the user to turn the power on at a specific time and turn the power off after the lapse of a set time interval. Thus, a user can leave the yoga mat on the floor of his/her house or apartment connected to a wall outlet knowing that the mat will be comfortable to use when he/she wakes up in the morning and starts to use the mat, particularly on a cold winter mourning. Also, the user can set the timer for the duration, or perhaps for the first few minutes, of a class or workout session without fear of leaving the pad on for extended periods of time.

In FIG. 5, a cross-sectional view of the infrared heating element 126 shows the element sandwiched between a sheet of aluminum foil 122 and a silver pad 128. The silver pad, in a preferred embodiment, may be a combination of foil and non-woven synthetic materials which serve to direct the heat from the heating element 126 upwards for absorption by the user, instead of downwards into the floor. This improves the efficiency of the infrared heat exercise pad and allows for the minimization of energy consumption while maintaining comfort and heat for the user.

Heating wire 126 can be a single continuous loop of heating wire, or two to four loops of heating wires which are connected in parallel or in series. In an embodiment the heating wire(s) are connected directly to the power control device and includes one or more thermostats 168.

In a preferred embodiment it should be noted that the heating elements 126 are located between the layers 122 and 128 of the mat and supply heat equally to each layer, the infrared heat exercise pad does not have a top or a bottom but can be used with either side of the pad being the top side. If desired, the pad can be placed within an enclosure which may be made of a woven or unwoven material or a vinyl which can be colored, such as black, orange, purple, pink, green, blue, yellow, etc., or can have multiple colors which may include a pleasing design. One or both surfaces of the enclosure may be treated with a slip resistant material such as a rubberized coating to help prevent the infrared heat exercise pad from sliding on the floor and/or the user from sliding off the mat. In a preferred embodiment, the electrical wiring circuit may have multiple separate heating zones. Each heating zone includes a flexible heating wire which can have a flexible coating of insulation of, for example, PVC or the like. Also, each zone can have its own controllable thermostat within power control device 104. The multiple individual heating elements and the multiple thermostats of the separate zones may be connected in parallel with each other and each zone is connected to power control device 104 and may include a controllable timer 166. Alternatively, as noted above, in another embodiment, the heating coil wire is connected as a single zone either with a thermostat or without a thermostat. When the heating wires are not connected to a thermostat, the heating wires are designed to warm the infrared heat exercise pad to a temperature of between forty-one degrees and forty-five degrees Celsius (41° to 45° C.).

In operation, and specifically in the winter or on cold days, the infrared heat exercise pad can be connected to a source of electrical power by inserting the plug into a wall outlet and setting the timer 166 to supply electrical power to the infrared heat exercise pad at a specified time such as, for example, twenty minutes before the user plans on using the mat. As this is not the first time that the mat is being used, if the yoga mat has one or more thermostats, the thermostat(s) will have been previously set by the user to warm each individual zone to a desired temperature.

It is to be appreciated that when multiple thermostats are used, the various thermostats can be set to warm the pad to a uniform temperature, or they can be set to warm one or more zones to different temperatures. When the timer begins to supply power through the power control device 104, each thermostat 168 begins to supply power to its zone until the set temperature at the temperature sensor 180 is reached, at which time the thermostat 168 stops the warming process. It is to be noted that some thermostats may supply power to its zone while other thermostats may be turned off. Thus, when the user of the mat starts to use the mat it is at a comfortable temperature and will not chill the user. When the timer 166 advances to its specific turn off time, or expires its count-down, all power to the various zones will be turned off.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated. All such modifications and variations are within the scope of the present invention as determined by the appended claims when interpreted in accordance with the breadth to which they are entitled.

I claim:

1. An infrared heat exercise pad, comprising:
an upper material layer;
a lower material layer;
a heat generating layer comprising a heating coil having an upper foil layer and a lower foil layer; and
a means for exciting said heating coil.

2. The infrared heat exercise pad of claim 1, further comprising:
a padding layer.

3. The infrared heat exercise pad of claim 1, wherein said padding layer further comprises a foam padding

4. The infrared heat exercise pad of claim 3, further comprising:
an intermediate material layer disposed between said padding layer and said lower foil layer.

5. The infrared heat exercise pad of claim 1, wherein said means for exciting said heating coil further comprises a DC power source.

6. The infrared heat exercise pad of claim 1, wherein said means for exciting said heating coil further comprises an AC power source.

7. The infrared heat exercise pad of claim 1, wherein said foil layer comprises aluminum foil.

8. The infrared heat exercise pad of claim 1, wherein said material layers comprise canvas.

9. The infrared heat exercise pad of claim 1, wherein said padding layer comprises cotton.

10. The infrared heat exercise pad of claim 1, wherein said padding layer comprises foam.

11. An infrared heat exercise pad, comprising:
a lower material layer;
a padding layer;
an intermediate material layer;
a lower foil layer;
a heating coil;
an upper foil layer;
an upper material layer; and
a means to bind said layers together.

12. The infrared heat exercise pad of claim 11, further comprising:
a power control device in electrical connection with said heating coil; and
a power supply in electrical connection with said power control device.

13. The infrared heat exercise pad of claim 12, wherein said power control device further comprises a display for displaying a temperature mode, a heating mode and a time.

14. The infrared heat exercise pad of claim 11, further comprising a means for adjusting the temperature of said heating coil.

15. The infrared heat exercise pad of claim 14, wherein the means for adjusting the temperature of said heating coil comprises a thermostat.

16. The infrared heat exercise pad of claim 15, wherein the means for adjusting the temperature of said heating coil further comprises a temperature sensor in electrical communication with said thermostat.

17. The infrared heat exercise pad of claim 11, further comprising a means for adjusting the duration for heating said heating coil.

18. The infrared heat exercise pad of claim 17, wherein said means for adjusting the duration for heating said heating coil comprises a timer.

19. The infrared heat exercise pad of claim 11, wherein said foil layer comprises aluminum foil.

21. The infrared heat exercise pad of claim 11, wherein said material layers comprise canvas.

22. The infrared heat exercise pad of claim 11, wherein said padding layer comprises foam.

23. The infrared heat exercise pad of claim 11, further comprising a perimetric binding attaching said upper material layer to said lower material layer.

24. The infrared heat exercise pad of claim 11, further comprising an adhesive.
